(19) United States
(12) Patent Application Publication
(10) Pub. No.: US 2025/0029724 A1
(43) Pub. Date: Jan. 23, 2025

(54) DEVICE AND METHOD FOR PRIVACY-PRESERVING ECG DATA COLLECTION FOR ARRHYTHMIA CLASSIFICATION

(71) Applicant: KOREA UNIVERSITY RESEARCH AND BUSINESS FOUNDATION, Seoul (KR)

(72) Inventors: Yon Dohn CHUNG, Seoul (KR); Hyubjin LEE, Seoul (KR); Minsoo KIM, Seoul (KR)

(21) Appl. No.: 18/777,984

(22) Filed: Jul. 19, 2024

(30) Foreign Application Priority Data
Jul. 21, 2023 (KR) 10-2023-0095129

(51) Int. Cl. G16H 50/20; A61B 5/349; G06N 3/045; G06N 3/084

(52) U.S. Cl. CPC G16H 50/20; A61B 5/349; G06N 3/045; G06N 3/084

(57) ABSTRACT

Disclosed is a privacy-preserving electrocardiogram (ECG) data collecting method for arrhythmia classification. The privacy-preserving ECG data collecting method is performed by a computing device including at least a processor and includes training a feature extraction model, a personal identification model, and an arrhythmia classification model using learning data that includes ECG data with a predetermined length; and training a noise model, and the feature extraction model receives the ECG data as input and outputs ECG features, the personal identification model receives the ECG features as input and identifies an individual corresponding to the ECG features, the arrhythmia classification model receives the ECG features as input and classifies arrhythmia corresponding to the ECG features, and the noise model generates noise with the same length as that of the ECG features.

### CROSS-REFERENCE TO RELATED APPLICATION(S)

This application claims the benefit under 35 USC § 119 (a) of Korean Patent Application No. 10-2023-0095129 filed on Jul. 21, 2023 in the Korean Intellectual Property Office, the entire disclosure of which is incorporated herein by reference for all purposes.

### BACKGROUND

### 1. Field

At least one example embodiment relates to a method of preventing privacy breach that may occur when collecting electrocardiogram (ECG) data for arrhythmia classification, and more particularly, to a method of preventing personal identification by adding noise since collecting ECG data with personal identification ability in its original form may lead to privacy breach.

### 2. Description of Related Art

Digital healthcare measures and collects a user's electrocardiogram (ECG) using a device, such as a smartwatch, for a personalized health management service. ECG that is an electrical signal generated from the activity of the heart may be used to detect and classify a cardiac disease, such as arrhythmia, and to identify an individual since ECG data has unique characteristics for each individual.

Meanwhile, the accuracy of arrhythmia classification or personal identification of ECG data is increasing through deep learning technology. In particular, attention mechanism is one of deep learning technologies and allows a model to focus on important information by assigning a weight to an important part of input data. The use of this attention mechanism has significantly improved the accuracy of both arrhythmia classification and personal identification using ECG data.

Since ECG data has unique characteristics for each individual, collecting the ECG data in the original form may lead to privacy breach. Personal identification may be prevented by simply adding noise to ECG data to prevent privacy breach. However, if noise is added to the entire data, usability such as arrhythmia classification may be lowered. Accordingly, noise needs to be added to ECG data such that the added noise only interferes with personal identification and does not interfere with arrhythmia classification.

### SUMMARY

A technical subject of at least one example embodiment is to provide a method and device for privacy-preserving electrocardiogram (ECG) data collection for arrhythmia classification.

A privacy-preserving ECG data collecting method according to an example embodiment refers to a privacy-preserving ECG data collecting method performed by a computing device including at least a processor, and includes training a feature extraction model, a personal identification model, and an arrhythmia classification model using learning data that includes ECG data with a predetermined length; and training a noise model, and the feature extraction model receives the ECG data as input and outputs ECG features, the personal identification model receives the ECG features as input and identifies an individual corresponding to the ECG features, the arrhythmia classification model receives the ECG features as input and classifies arrhythmia corresponding to the ECG features, and the noise model generates noise with the same length as that of the ECG features.

Also, a privacy-preserving ECG data collecting method according to another example embodiment refers to a privacy-preserving ECG data collecting method performed by a computing device including at least a processor, and includes receiving target ECG data; extracting ECG features corresponding to the target ECG data; generating attention distribution for noise; and generating the noise-added ECG features.

According to a method and device for privacy-preserving ECG data collection for arrhythmia classification, when performing arrhythmia classification or personal identification on ECG data using deep learning attention mechanism, it is possible to obtain information on a necessary part and to add strong noise to a part necessary for the personal identification and to add small noise to a part necessary for the arrhythmia classification in the ECG data based on the obtained information.

In this manner, although data leakage occurs, an individual may not be identified and privacy may be protected. Also, although the arrhythmia classification is performed using the ECG data with noise added, it is possible to prevent great degradation in classification performance.

### BRIEF DESCRIPTION OF THE DRAWINGS

These and/or other aspects, features, and advantages of the disclosure will become apparent and more readily appreciated from the following description of example embodiments, taken in conjunction with the accompanying drawings of which:

FIG. 1 illustrates a structure of a first deep learning model according to an example embodiment;

FIG. 2 illustrates a structure of a second deep learning model according to an example embodiment;

FIG. 3 illustrates a training process of the second deep learning model shown in FIG. 2; and

FIG. 4 is a flowchart illustrating a privacy-preserving electrocardiogram (ECG) data collecting method according to an example embodiment.

### DETAILED DESCRIPTION

The aforementioned features and effects of the disclosure will be apparent from the following detailed description related to the accompanying drawings and accordingly those skilled in the art to which the disclosure pertains may easily implement the technical spirit of the disclosure.

Various modifications and/or alterations may be made to the disclosure and the disclosure may include various example embodiments. Therefore, some example embodiments are illustrated as examples in the drawings and described in detailed description. However, they are merely intended for the purpose of describing the example embodiments described herein and may be implemented in various forms. Therefore, the example embodiments are not construed as limited to the disclosure and should be understood to include all changes, equivalents, and replacements within the idea and the technical scope of the disclosure.

Although terms of “first,” “second,” and the like are used to explain various components, the components are not limited to such terms. These terms are used only to distinguish one component from another component.

For example, a first component may be referred to as a second component, or similarly, the second component may be referred to as the first component within the scope of the present disclosure. As used herein, the term “and/or” includes any and all combinations of one or more of the associated listed items.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Hereinafter, example embodiments will be described with reference to the accompanying drawings. However, the scope of the patent application is not limited to or restricted by such example embodiments. Like reference numerals used herein refer to like elements throughout.

Herein, proposed is a method of preventing personal identification from electrocardiogram (ECG) data by adding appropriate noise to ECG while maintaining data usability for arrhythmia classification. One approach to privacy protection is adding random noise to ECG features. However, the added noise disrupts personal identification, but may also degrade performance of the arrhythmia classification. Accordingly, usefulness of data is reduced due to the added noise. Therefore, it is necessary to determine which part of the ECG features is used for the personal identification and which part is used for the arrhythmia classification. Then, it is possible to generate appropriate noise and to prevent the personal identification without degrading the performance of the arrhythmia classification.

From this perspective, the present invention proposes two deep learning models using attention mechanism. A first model may be referred to as an attention mapper and provides information on parts of the ECG features to focus on during identification and classification operations. The first model may perform identification and classification operations using the attention mechanism, after extracting features with a feature extraction model (e.g., residual neural network (ResNet)) and may generate information on which part of the ECG features needs to be focused on during each task. A second model may be referred to as a noise generator and generates appropriate noise and adds the generated noise to the ECG features to interfere with only identification performance without degrading classification performance. The second model uses information generated by the first model on which parts of ECG features demand attention for identification and classification, respectively. The second model maximizes noise in parts of the ECG features necessary for the identification and minimizes noise in parts necessary for the classification. Hereinafter, the proposed two models are described in more detail.

### First Model (Attention Mapper)

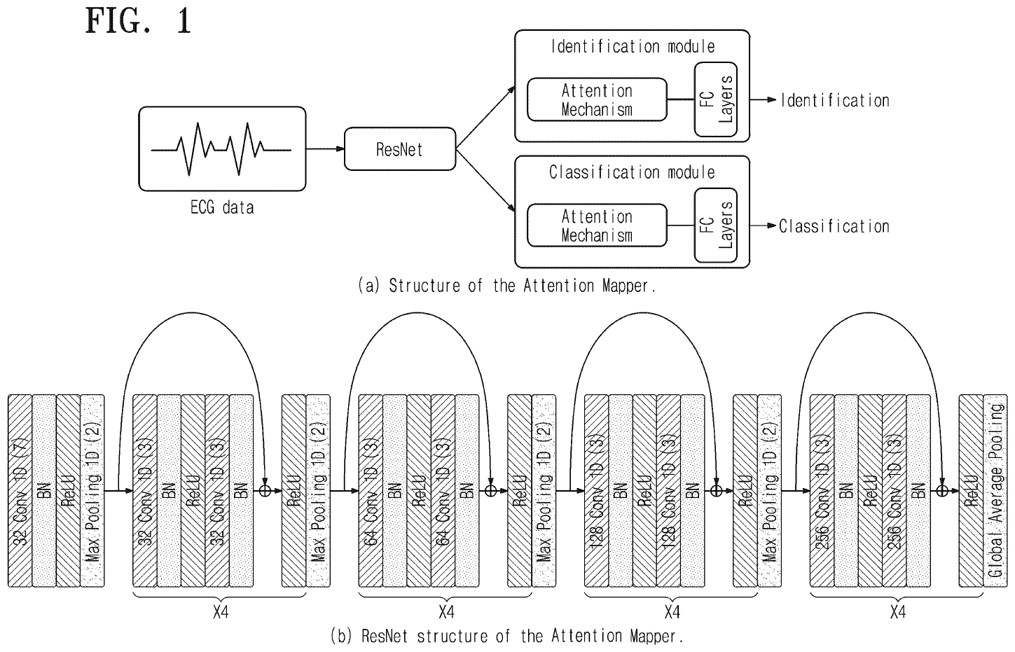

The first model, the attention mapper, is a deep learning model that simultaneously performs a personal identification task and an arrhythmia classification task from ECG data. A structure of the attention mapper is illustrated in (a) of FIG. 1. Initially, features are extracted from the ECG data by stacking multiple 1D convolutional neural network (CNN) layers. In the attention mapper, residual neural network (ResNet) may be used to prevent degradation in learning performance due to a vanishing gradient or exploding gradient issue. However, the scope of the present invention is not limited thereto and, depending on example embodiments, an arbitrary feature extraction technique may be used or an arbitrary artificial neural network (ANN) model may be used. According to an example embodiment, as shown in (b) of FIG. 1, the ResNet structure may be combined Rectified Linear Unit (ReLU) activation function and batch normalization (BN), to facilitate training. In (b) of FIG. 1, n ConV 1D (m) refers to a 1D CNN operation that utilizes n filters with size m. To resize features during the ResNet process, max pooling that compares two values and halves a length by maintaining a larger value may be used. Finally, global average pooling may be used to compute average for each channel and to output ECG features with a channel length. However, the optimal ResNet structure may be changed depending on a length of input ECG data.

ECG features extracted in a vector form through ResNet are used for personal identification and arrhythmia classification in the respective corresponding modules. As shown in (a) of FIG. 1, an identification module and a classification module have the same structure. That is, the identification module and the classification module may include attention mechanism and fully connected (FC) layers. The attention mechanism refers to a technique that focuses on a significant part of input to improve performance when solving a problem. In each module, the attention mechanism focuses on important parts of ECG features for the personal identification and the arrhythmia classification, respectively. Here, various attention mechanisms may be used. According to an example embodiment, Transformer's self-attention (A. Vaswani, N. Shazeer, N. Parmar, J. Uszkoreit, L. Jones, A. N. Gomez, Ł. Kaiser, I. Polosukhin, Attention is all you need, Advances in neural information processing systems 30 (2017).) may be used. After the attention mechanism, the ECG features may go through simple FC layers to produce output of the personal identification and the arrhythmia classification. Since the following noise generator uses information generated by the identification module and the classification module, the attention mapper, that is, a feature extraction model, a personal identification model, and an arrhythmia classification model, needs to be pre-trained.

### Second Model (Noise Generator)

The second model, which may also be referred to as the noise generator, aims to generate ECG features with noise that prevents personal identification while preserving ECG data's usefulness for arrhythmia classification. A noise model in the noise generator may have a multi-layer perceptron (MLP) structure with three hidden layers and ReLU activation functions and may output a vector with the same size as that of an input vector. The noise model receives the ECG features extracted by ResNet as input and generates noise with the same length as that of the extracted ECG features. However, this simple structure may be insufficient to generate desired noise. Therefore, the attention mechanism in the identification module and the classification module of the pre-trained attention mapper may be used. Considering that the input ECG features have the length of n, attention distribution generated during a processing of the attention mechanism is an n×n matrix with values summing to 1 in each row. This matrix generated by each module indicates which parts of the ECG features are significant for identification and classification, respectively. That is, a larger value represents a more significant ECG part in a corresponding task. ECG features in a column of each matrix may be column-wisely summed to form a vector with the size of n, which is then used as the attention distribution for noise generation.

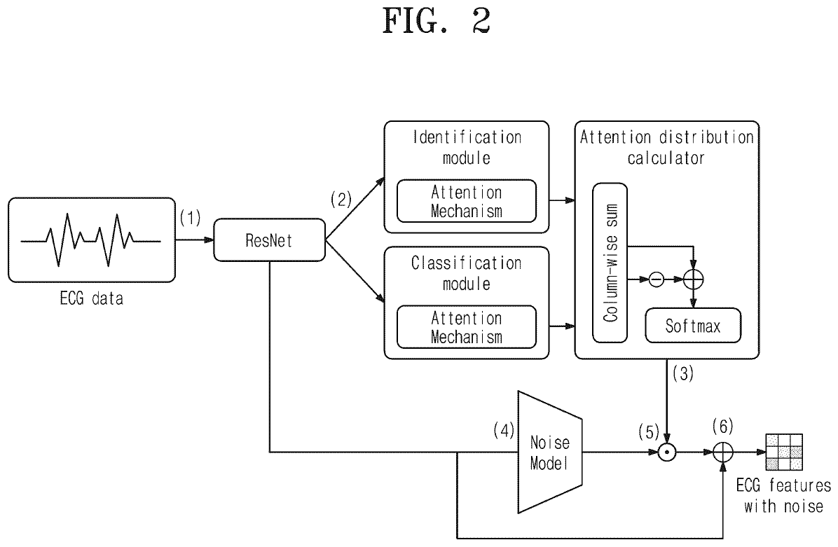

As shown in FIG. 2, the noise generator utilizes the noise model and the attention distribution to generate appropriate noise. In more detail, the noise generator (1) extracts features in a vector form from ECG data using the feature extraction model (e.g., ResNet) of the pre-trained attention mapper, (2) inputs the ECG features to the attention mechanism of the identification module (or personal identification model) and the classification module (or arrhythmia classification model) of the attention mapper to compute the attention distribution for each task, and (3) column-wisely sums each attention distribution aᵢ and a_{c} to generate two vectors and subtracts a vector for arrhythmia classification from a vector for personal identification. That is, the vector for the arrhythmia identification is subtracted from the vector for the personal identification. The softmax function is applied to results and new attention distribution for noise may be generated. In the case of applying the softmax function, a sum of values included in the vector may be converted to become 1. That is, the attention distribution may be generated by computing softmax(sum_{column-wise}(aᵢ)−sum_{column-wise}(a_{c})). In the attention distribution, a value is larger in a part which classification does not focus while the identification focuses. The attention distribution may be understood to represent a part in which noise may be added to disturb only identification performance without degrading classification performance. (4) The noise model receives the ECG features extracted by the feature extraction model (e.g., ResNet) as input and outputs noise with the same length. The same length may indicate that a length of input ECG data and/or length of extracted ECG features are the same. (5) A weighted sum is computed by taking the element-wise product of the generated noise and the generated attention distribution. That is, the weighted noise may be generated. (6) Finally, noise-added ECG features may be generated by taking an element-wise sum of the ECG features and the results (which may indicate the weighted noise derived in the process of (5)). Through this, noise may be added to the ECG features and the generated noise-added ECG features may be used for collection.

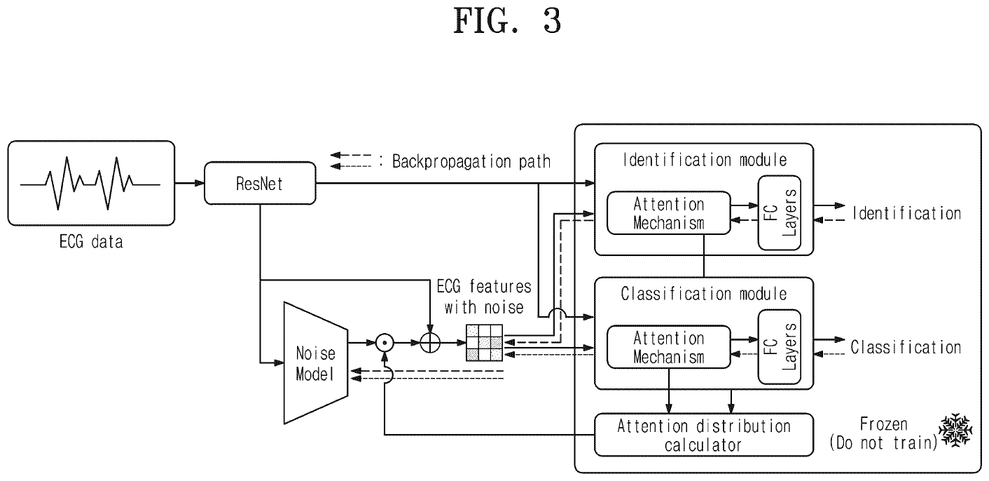

A training process of the noise generator is as shown in FIG. 3. The ECG features with noise generated by the noise generator are input to the identification module and the classification module of the attention mapper. The noise model is trained using a backpropagation algorithm to minimize identification accuracy and to maximize classification accuracy. As a result of this training process, the noise generator may generate ECG features with noise that has lower identification accuracy but higher classification accuracy. Also, depending on example embodiments, similar to transfer learning and/or fine-tuning techniques in which some layers or the entire model of the pre-trained model are frozen during the training process to prevent weight update, the noise generator may train only the noise model without training models in the feature extraction model (e.g., ResNet), the identification module, and the classification module.

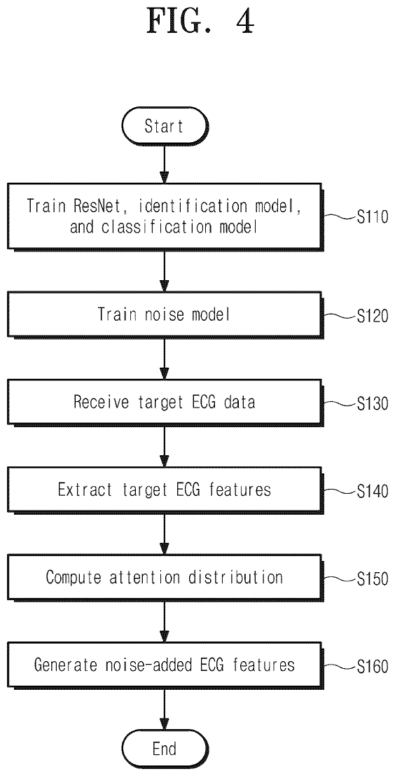

FIG. 4 is a flowchart illustrating a privacy-preserving ECG data collecting method according to an example embodiment. Depending on example embodiments, the privacy-preserving ECG data collecting method may also be referred to as a privacy-preserving ECG data generation model generating method, a privacy ECG data generating method, and the like. The ECG data collecting method may be performed by an ECG data collecting device implemented as a computing device that includes at least a processor and/or a memory. Likewise, the ECG data collecting device may also be referred to as an ECG data generation model generating device, an ECG data generating device, and the like. At least a portion of operations included in the ECG data collecting method may be understood as an operation of the processor included in the computing device. Also, the computing device may include a personal computer (PC), a server, a laptop computer, a tablet PC, a notebook, a smartphone, a smart watch, a head mounted device (HMD), a smart ring, and smart glasses. Hereinafter, when describing the ECG data collecting method, further description related to overlapping contents of the foregoing description is omitted.

Initially, in operation S110, training of a feature extraction model, a (personal) identification model, and an (arrhythmia) classification model is performed. The feature extraction model refers to a model that receives ECG data (, which may indicate an ECG signal) as input and extracts features (ECG features) in a vector form corresponding to the ECG data. For example, the feature extraction model may be ResNet. The identification model refers to a model that receives the extracted ECG features as input and identifies an individual corresponding to the ECG features. The classification model refers to a model that receives the extracted ECG features as input and classifies arrhythmia corresponding to the ECG features. The identification model and the classification model may be attention networks having the same structure. Also, ECG data that constitutes learning data may be preprocessed to a have a predetermined length.

In operation S120, a noise model is trained. In a process of training the noise model, the pre-trained feature extraction model, identification model, and classification model are frozen (i.e., not trained) and only the noise model is trained.

In more detail, the ECG features (here, ECG features that are extracted by the feature extraction model) with noise generated by a noise generator (or noise model) are input to the identification model and the classification model. Here, the noise model is trained using a backpropagation algorithm to minimize identification accuracy and to maximize classification accuracy. As a result of this training, the noise model may generate noise that has lower identification accuracy but higher classification accuracy. Also, the noise model may refer to an arbitrary neural network model.

In operation S130, target ECG data (ECG signal) is received. The target ECG data may be received from an external device through a wired/wireless communication network or may also be received from an ECG measurement device implemented in an ECG collection device. Also, the target ECG data may have a predetermined length or may be preprocessed to have the predetermined length.

In operation S140, target ECG features corresponding to the target ECG data are extracted. The target ECG features may be extracted by inputting the target ECG data to the trained feature extraction model (e.g., ResNet).

In operation S150, attention distribution for noise is computed. To this end, the extracted target ECG features are input to the trained identification model and the trained classification model. Through this, attention distribution for identification (first attention distribution) and attention distribution for classification (second attention distribution) are generated. To generate the attention distribution for noise, the first attention distribution and the second attention distribution in a matrix form are converted to a vector form. A weight to be multiplied by noise may be generated by subtracting the second attention distribution converted to the vector form from the first attention distribution converted to the vector form. That is, the attention distribution for noise may be understood as the weight to be multiplied by noise. Here, the softmax function may be applied to the subtraction results of the attention distribution.

In operation S160, the noise-added ECG features are generated. In more detail, the noise model may receive the extracted target ECG features and may generate noise with the same length as that of the target ECG features. The generated noise may be multiplied by the weight and then added to the target ECG features to generate the noise-added ECG features.

The aforementioned ECG data collecting method includes all of a model training process and a process of generating noise-added ECG features using trained models. However, depending on example embodiments, the ECG data collecting method may also be implemented to include only the process of generating the noise-added ECG features using the trained models. Since the ECG data collecting method in this case uses the trained models, only operations S130 to S160 may be included.

The aforementioned method according to example embodiments may be implemented in a form of a program executable by a computer apparatus. Here, the program may include, alone or in combination, a program instruction, a data file, and a data structure. The program may be specially designed to implement the aforementioned method or may be implemented using various types of functions or definitions known to those skilled in the computer software art and thereby available. Also, here, the computer apparatus may be implemented by including a processor or a memory that enables a function of the program and, if necessary, may further include a communication apparatus.

The program for implementing the aforementioned method may be recorded in computer-readable record media. The media may include, for example, a semiconductor storage device such as an SSD, ROM, RAM, and a flash memory, magnetic disk storage media such as a hard disk and a floppy disk, optical record media such as disc storage media, a CD, and a DVD, magneto optical record media such as a floptical disk, and at least one type of physical device capable of storing a specific program executed according to a call of a computer such as a magnetic tape.

Although some example embodiments of an apparatus and method are described, the apparatus and method are not limited to the aforementioned example embodiments. Various apparatuses or methods implementable in such a manner that one of ordinary skill in the art makes modifications and alterations based on the aforementioned example embodiments may be an example of the aforementioned apparatus and method. For example, although the aforementioned techniques are performed in order different from that of the described methods and/or components such as the described system, architecture, device, or circuit may be connected or combined to be different form the above-described methods, or may be replaced or supplemented by other components or their equivalents, it still may be an example embodiment of the apparatus and method.

The device described above can be implemented as hardware elements, software elements, and/or a combination of hardware elements and software elements. For example, the device and elements described with reference to the embodiments above can be implemented by using one or more general-purpose computer or designated computer, examples of which include a processor, a controller, an ALU (arithmetic logic unit), a digital signal processor, a microcomputer, an FPGA (field programmable gate array), a PLU (programmable logic unit), a microprocessor, and any other device capable of executing and responding to instructions. A processing device can be used to execute an operating system (OS) and one or more software applications that operate on the said operating system. Also, the processing device can access, store, manipulate, process, and generate data in response to the execution of software. Although there are instances in which the description refers to a single processing device for the sake of easier understanding, it should be obvious to the person having ordinary skill in the relevant field of art that the processing device can include a multiple number of processing elements and/or multiple types of processing elements. In certain examples, a processing device can include a multiple number of processors or a single processor and a controller. Other processing configurations are also possible, such as parallel processors and the like.

The software can include a computer program, code, instructions, or a combination of one or more of the above and can configure a processing device or instruct a processing device in an independent or collective manner. The software and/or data can be tangibly embodied permanently or temporarily as a certain type of machine, component, physical equipment, virtual equipment, computer storage medium or device, or a transmitted signal wave, to be interpreted by a processing device or to provide instructions or data to a processing device. The software can be distributed over a computer system that is connected via a network, to be stored or executed in a distributed manner. The software and data can be stored in one or more computer-readable recorded medium.

A method according to an embodiment of the invention can be implemented in the form of program instructions that may be performed using various computer means and can be recorded in a computer-readable medium. Such a computer-readable medium can include program instructions, data files, data structures, etc., alone or in combination. The program instructions recorded on the medium can be designed and configured specifically for the present invention or can be a type of medium known to and used by the skilled person in the field of computer software. Examples of a computer-readable medium may include magnetic media such as hard disks, floppy disks, magnetic tapes, etc., optical media such as CD-ROM's, DVD's, etc., magneto-optical media such as floptical disks, etc., and hardware devices such as ROM, RAM, flash memory, etc., specially designed to store and execute program instructions. Examples of the program instructions may include not only machine language codes produced by a compiler but also high-level language codes that can be executed by a computer through the use of an interpreter, etc. The hardware mentioned above can be made to operate as one or more software modules that perform the actions of the embodiments of the invention and vice versa.

While the present invention is described above referencing a limited number of embodiments and drawings, those having ordinary skill in the relevant field of art would understand that various modifications and alterations can be derived from the descriptions set forth above. For example, similarly adequate results can be achieved even if the techniques described above are performed in an order different from that disclosed, and/or if the elements of the system, structure, device, circuit, etc., are coupled or combined in a form different from that disclosed or are replaced or substituted by other elements or equivalents. Therefore, various other implementations, various other embodiments, and equivalents of the invention disclosed in the claims are encompassed by the scope of claims set forth below.

What is claimed is:

1. A method for privacy-preserving electrocardiogram (ECG) data collection performed by a computing device comprising at least a processor, the method comprising:
training a feature extraction model, a personal identification model, and an arrhythmia classification model using learning data that includes ECG data with a predetermined length; and
training a noise model,
wherein the feature extraction model receives the ECG data as input and outputs ECG features,
the personal identification model receives the ECG features as input and identifies an individual corresponding to the ECG features,
the arrhythmia classification model receives the ECG features as input and classifies arrhythmia corresponding to the ECG features, and
the noise model generates noise with the same length as that of the ECG features.

2. The method of claim 1, wherein the feature extraction model is residual neural network (ResNet), and
the personal identification model and the arrhythmia classification model are attention networks having the same structure.

3. The method of claim 1, wherein the training of the noise model comprises training the noise model while freezing the feature extraction model, the personal identification model, and the arrhythmia classification model, and
the training of the noise model comprises:
extracting the ECG features using the feature extraction model;
generating first attention distribution that is attention distribution for personal identification by inputting the ECG features to the personal identification model;
generating second attention distribution that is attention distribution for arrhythmia classification by inputting the ECG features to the arrhythmia classification model;
converting the first attention distribution and the second attention distribution to a vector form;
generating attention distribution for noise by subtracting the second attention distribution converted to the vector form from the first attention distribution converted to the vector form;
generating noise having the same length as that of the ECG features using the noise model;
generating weighted noise by multiplying the generated noise by the attention distribution for noise; and
generating the noise-added ECG features by adding the weighted noise and the ECG features.

4. The method of claim 3, wherein the noise model is trained using backpropagation to minimize personal identification accuracy and to maximize arrhythmia classification accuracy.

5. A method for privacy-preserving electrocardiogram (ECG) data collection, performed by a computing device comprising at least a processor, the method comprising:
receiving target ECG data;
extracting ECG features corresponding to the target ECG data;
generating attention distribution for noise; and
generating the noise-added ECG features.

6. The method of claim 5, wherein the extracting of the ECG features is performed using pre-trained residual neural network (ResNet).

7. The method of claim 5, wherein the generating of the attention distribution for noise comprises:
generating first attention distribution that is attention distribution for personal identification by inputting the target ECG features to a pre-trained personal identification model;
generating second attention distribution that is attention distribution for arrhythmia classification by inputting the target ECG features to a pre-trained arrhythmia classification model;
converting the first attention distribution and the second attention distribution in a matrix form to a vector form; and
generating the attention distribution for noise by subtracting the second attention distribution converted to the vector form from the first attention distribution converted to the vector form.

8. The method of claim 7, wherein the generating of the noise-added ECG features comprises:
generating noise with the same length as that of the target ECG features;
generating weighted noise by multiplying the generated noise by the attention distribution for noise; and
generating the noise-added ECG features by adding the weighted noise and the ECG features.
